(19) United States
(12) Patent Application Publication
(10) Pub. No.: US 2023/0172218 A1
(43) Pub. Date: Jun. 8, 2023

(54) Facilitating Traceability of Animal-Based Meat Products

(71) Applicant: Prox Animal Health, LLC, Fort Worth, TX (US)

(72) Inventor: Barry Beverly, Fort Worth, TX (US)

(21) Appl. No.: 18/061,445

(22) Filed: Dec. 3, 2022

Related U.S. Application Data
(60) Provisional application No. 63/264,918, filed on Dec. 3, 2021.

(51) Int. Cl. A22C 17/10; G06Q 10/087; A22B 5/00; A22C 17/00

(52) U.S. Cl. CPC A22C 17/10; G06Q 10/087; A22B 5/007; A22C 17/0093

(57) ABSTRACT

In certain embodiments, a non-transitory computer-readable storage medium stores a program for execution by one or more processors that includes instructions for receiving a label-generating request for generating a machine-readable image file for an animal-based meat product that corresponds to a particular source animal. The program further includes instructions for accessing, in response to receiving the label-generating request, digital animal records for source animals to identify a digital animal record for the particular source animal. The digital animal records include animal information that includes identification information, location information, and health information for the source animals. The program further includes instructions for generating, based on the animal information of the digital animal record for the particular source animal, a machine-readable image file for the particular source animal that, when decoded, makes available meat facts regarding the particular source animal, and for transmitting the machine-readable image file to a requesting device.

### CROSS-REFERENCE TO RELATED APPLICATIONS

This application claims the benefit of U.S. Provisional Application No. 63/264,918, filed on Dec. 3, 2021, which is incorporated by reference.

### TECHNICAL FIELD

This disclosure relates generally to software, and, in particular embodiments, to facilitating traceability of animal-based meat products.

### BACKGROUND

Among the foods consumed by many humans are animal-based meat products. Animal-based meat products frequently are sourced from animals raised on family or commercial farms. The manner in which these source animals are raised and the health of these source animals may affect the quality of meat products sourced from these source animals.

### SUMMARY

In certain embodiments, a non-transitory computer-readable storage medium stores a program for execution by one or more processors that includes instructions for receiving a label-generating request for generating a machine-readable image file for an animal-based meat product that corresponds to a particular source animal. The program further includes instructions for accessing, in response to receiving the label-generating request, digital animal records for source animals to identify a digital animal record for the particular source animal. The digital animal records include animal information that includes identification information, location information, and health information for the source animals. The program further includes instructions for generating, based on the animal information of the digital animal record for the particular source animal, a machine-readable image file for the particular source animal that, when decoded, makes available meat facts regarding the particular source animal, and for transmitting the machine-readable image file to a requesting device.

In certain embodiments, a method includes decoding, by a user device, data coded in an optical scan of a physical representation of a machine-readable image file located on a label for an animal-based meat product. The method further includes accessing, by the user device according to the data coded in the optical scan, meat facts about a particular source animal corresponding to the animal-based meat product. The method further includes displaying, by the user device, the meat facts about the particular source animal associated with the animal-based meat product.

In certain embodiments, a system includes a storage system that stores digital animal health records for animals, the digital animal health records including identifying information and health records for the animals. The system further includes one or more processors and a non-transitory computer-readable storage medium storing a program for execution by the one or more processors. The program includes instructions for receiving a request to generate a first code for identifying a source animal associated with an animal-based meat product. The program includes instructions for accessing, from digital animal records for animals, a digital animal record corresponding to the source animal associated with the animal-based meat product. The program includes instructions for generating, based on the digital animal record corresponding to the source animal associated with the animal-based meat product, a first code for identifying the source animal, and for. The program includes instructions for updating the digital animal record corresponding to the source animal associated with the animal-based meat product with the first code for identifying the source animal, and for transmitting the first code to a receiving device.

### BRIEF DESCRIPTION OF THE DRAWINGS

For a more complete understanding of this disclosure, and the advantages thereof, reference is now made to the following descriptions taken in conjunction with the accompanying drawings, in which:

FIG. 1 illustrates an example system for facilitating the traceability of animal-based meat products, according to certain embodiments;

FIG. 2 illustrates an example system for facilitating the traceability of animal-based meat products, according to certain embodiments;

FIG. 3 illustrates additional details of an example processing system and storage unit, according to certain embodiments;

FIG. 4 illustrates additional details of an example user device, according to certain embodiments;

FIG. 5 illustrates additional details of example digital animal records, according to certain embodiments;

FIGS. 6A-6C illustrate example labels that include a physical representation of a digital image file for a source animal of an animal-based meat product, according to certain embodiments;

FIG. 7 illustrates an example method for facilitating the traceability of animal-based meat products, according to certain embodiments;

FIG. 8 illustrates an example method for facilitating the traceability of animal-based meat products, according to certain embodiments; and

FIG. 9 illustrates an example method for facilitating the traceability of animal-based meat products, according to certain embodiments.

### DETAILED DESCRIPTION OF ILLUSTRATIVE EMBODIMENTS

Over time purchasers of food products have become more interested in learning facts about the foods they consume. In the case of meat products in particular, purchasers of animal-based meat products may seek information regarding the health of the animals they consume, the location at which an animal was raised, or other information about the source animal for the animal-based meat products. As a result the ability to trace the origins of a source animal for animal-based meat products and obtain other information about the source animal is desirable.

The sources of the foods the purchasing public consumes is often difficult to determine. Conventionally, the vast majority of animal health procedures and records have been recorded by hand, using a notepad for example, which may be not only unreliable, inefficient, and insecure, but may lack an ability for the health record to travel with an animal. For example, at family or commercial farms that raise animals that serve as sources for animal-based meat products, paper records are the primary technique for tracking inventory information (e.g., an animal identifier and the like) and health information related to the source animals. This paper technique creates difficulty in tracking an animal from the farm and throughout processing of the source animal into animal-based meat products for sale at a butcher or other suitable store. Furthermore, such paper records are not conducive to generating an informative traceability label for food resulting from processing of the animal (e.g., beef from cattle or pork from swine that ends up on a grocery store shelf).

Certain embodiments of this disclosure provide digital animal records that store animal information regarding the source animal for animal-based meat products. These digital animal records may be updated using information supplied electronically by users associated with the farm, for example, and may include inventory information and health information for the source animals.

Certain embodiments provide an ability to, along the path from farm to meat processing plant to the store shelf, communicate with a processing system that stores or has access to the digital animal records for the source animals to request an identifier that can be used to track the carcass and its parts from the farm to the point of sale. The identifier can be provided in the form of a code. For example, the code can be a physical representation of a digital image file, such as a quick resource (QR) code, a barcode, or the like.

Additionally or alternatively, certain purchasers of meat products may desire greater access to facts associated with the animal-based meat products that are being purchased. Certain embodiments are able to generate an enhanced meat packaging label that provides additional information. The additional information may be based on digital animal records. In certain embodiments, the meat packaging label includes a code (e.g., a physical representation of a machine-readable image file) that can be scanned using a user device and that is configured to provide access to animal information, such as certain meat facts associated with the animal-based meat product being purchased and/or the source animal for the animal-based meat product. In other words, the code may provide access to additional information about the animal-based meat product and/or the associated source animal. The additional information may be encoded in the code (e.g., the QR code), or the code may direct the user device to a website that provides the additional information to the user based on identifying information (e.g., for the source animal) included in a request generated from a scan of the code.

In certain embodiments, purchaser data, such as credit card information or loyalty program information, may be received from stores and added to the digital animal records. This purchaser data may be linked to the identifier associated with the animal-based meat product and/or the source animal, so that the purchaser and a particular meat product are linked in the digital animal records. In the event a foodborne illness is contracted by a purchaser of a particular animal-based meat product, using an identifier for the user, the source of the animal-based meat product may be determined from the digital animal records, which may facilitate a recall or other suitable mitigating action.

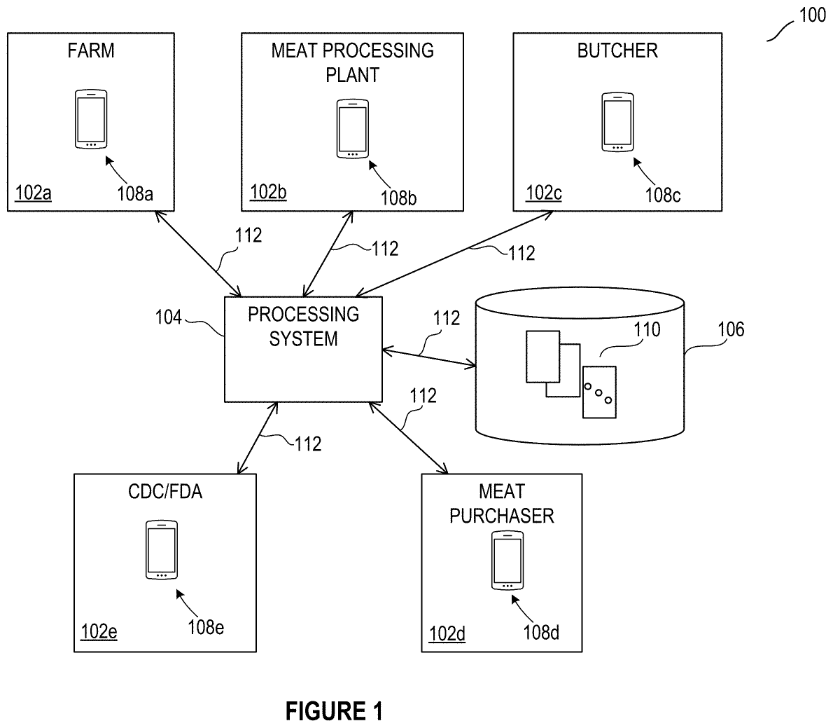

FIG. 1 illustrates an example system 100 for facilitating the traceability of animal-based meat products, according to certain embodiments. In the illustrated example, system 100 includes entities 102a-102e, which may be referred to generally as entities 102, a processing system 104, and a storage unit 106.

Entities 102 may include one or more farms 102a, one or more meat processing plants 102b, one or more butchers 102c, one or more meat purchasers 102d, and one or more public health oversight entities 102e, referred to primarily in the singular for simplicity. In a progression of raising source animals to ultimately be used for meat products, the source animals and resulting animal-based meat products may traverse one or more of entities 102, as described below.

Farm 102a may represent any farm on which source animals may be raised. For example, farm 102a may be a family or commercial farm for raising source animals. The source animals may include any suitable type of animal that may serve as an animal-based meat product. As just a few examples, source animals may include pigs, cows, turkeys, chickens, sheep, or any other suitable type of source animal. In certain scenarios, animals may be delivered to a feedlot prior to being sent to a meat processing plant (e.g., meat processing plant 102b, described below). For purposes of this description, it will be assumed that such a feedlot is part of farm 102a and/or meat processing plant 102b; however, this disclosure contemplates a separate feedlot being used.

Meat processing plant 102b represents a facility for processing source animals. In certain embodiments, live source animals are delivered from farm 102a to meat processing plant 102b for slaughter and processing into an animal carcass. In certain embodiments, live source animals are slaughtered at farm 102a and delivered from farm 102a to meat processing plant 102b as an animal carcass. In certain embodiments, meat processing plant 102b provides the whole or a portion of animal carcass to a purchaser (e.g., to butcher 102c) or subdivides the animal carcass into one or more subparts for purchase by a purchaser (e.g., butcher 102c).

Butcher 102c may be an entity that buys a portion or all of a source animal carcass from meat processing plant 102b for subdividing into one or more animal-based meat products for purchase. For example, butcher 102c may be a butcher at a grocery store, a butcher operating an independent meat shop, or any other seller of animal-based meat products for purchase. In certain embodiments, butcher 102c may buy the animal-based meat products already formed from the source animal carcass (e.g., from meat processing plant 102b).

Purchaser 102d may be a person (e.g., an individual shopper at butcher 102c) or entity (e.g., a buyer for a restaurant or the like) that purchases animal-based meat products from butcher 102c. In some cases, purchaser 102d is likely to be the consumer of the animal-based meat product.

Public health oversight entity 102e may include the Center for Disease Control (CDC), the Food and Drug Administration (FDA), or any other entity that may have an interest in learning the location of a source animal of an animal-based meat product. Public health oversight entity 102e may be involved if a foodborne illness is encountered by a consumer of an animal-based meat product.

Each of entities 102 may be associated with one or more user devices 108. For example, farm 102a may be associated with one or more user devices 108a, meat processing plant 102b may be associated with one or more user devices 108b, butcher 102c may be associated with one or more user devices 108c, meat purchaser 102d may be associated with one or more user devices 108d, and public health oversight entity 102e may be associated with one or more user devices 108e. User devices 108a-108e may be referred to generally as user devices 108.

User devices 108 may include any suitable computing device(s) capable of running software applications and accessing network 112 (described below). User devices 108 may be implemented in any suitable combination of hardware, firmware, and software. User devices 108 may include one or more computer systems at one or more locations. Each computer system may include any appropriate input devices, output devices, mass storage media, processors, memory, or other suitable components for receiving, processing, storing, and communicating data. For example, each computer system may include an integrated circuit (IC), printed circuit board (PCB), personal computer, laptop computer, workstation, network computer, kiosk, wireless data port, personal data assistant (PDA), one or more Internet Protocol (IP) telephones, one or more cellular/smart phones, tablet computers, wearable devices (e.g., smartwatch), one or more processors within these or other devices, or any other suitable processing device. Certain ones of the above or other user devices 108 may be mobile devices, such that they may be readily hand-carried by a user.

As examples and not by way of limitation, user device 108 may be an embedded computer system, a system-on-chip (SOC), a single-board computer system (SBC) (e.g., a computer-on-module (COM) or system-on-module (SOM)), a desktop computer system, a laptop or notebook computer system, an interactive kiosk, a mainframe, a mesh of computer systems, a mobile telephone, a PDA, a server, another type of host, or a combination of two or more of these. Where appropriate, user device 108 may include one or more computer systems; be unitary or distributed; span multiple locations; span multiple machines; or reside in a cloud, which may include one or more cloud components in one or more networks.

System 100 also includes processing system 104, which includes or otherwise has access to a storage unit 106. Entities 102 may interact with processing system 104, using user devices 108 for example, to provide animal information regarding source animals to processing system 104, to request codes for source animals, to request animal information regarding source animals, or for other suitable purposes.

Processing system 104 may include any suitable computing device(s) operable to communicate with one or more user devices 108 through network 112 to facilitate managing animal health. Processing system 104 may be implemented in any suitable combination of hardware, firmware, and software. Processing system 104 may include one or more computer systems (e.g., one or more servers) at one or more locations. Each computer system may include any appropriate input devices, output devices, mass storage media, processors, memory, or other suitable components for receiving, processing, storing, and communicating data. Processing system 104 may include, for example, a file server, a domain name server, a proxy server, a web server, a computer workstation, or any other device operable to respond to requests for data from user devices 108 and/or manage storage and processing of data in storage unit 106. Processing system 104 may execute with any of the well-known MS-DOS, PC-DOS, OS-2, MAC-OS, WINDOWS, UNIX, or other appropriate operating systems, including future operating systems.

As examples and not by way of limitation, processing system 104 may be an embedded computer system, an SOC, an SBC (e.g., a COM or SOM), a desktop computer system, a laptop or notebook computer system, an interactive kiosk, a mainframe, a mesh of computer systems, a mobile telephone, a PDA, a server, another type of host, or a combination of two or more of these. Where appropriate, processing system 104 may include one or more computer systems; be unitary or distributed; span multiple locations; span multiple machines; or reside in a cloud, which may include one or more cloud components in one or more networks.

Processing system 104 may include one or more software applications that communicate with a software application running on user devices 108 and that facilitate creating, maintaining, and searching of digital animal records 110 in storage unit 106.

Storage unit 106 may include any number of tangible storage media. Additionally, all or part of storage unit 106 could reside locally within processing system 104 or could reside at a location external but accessible to processing system 104 (e.g., within removable media, and/or at an external database). In certain embodiments, storage unit 106 may take the form of a suitable combination of volatile and non-volatile memory including, without limitation, magnetic media, optical media, random access memory (RAM), read only memory (ROM), removable media, or any other suitable memory component. Where appropriate, RAM may be dynamic RAM (DRAM) or static RAM (SRAM). Moreover, where appropriate, this RAM may be single-ported or multi-ported RAM. This disclosure contemplates any suitable RAM. In certain embodiments, storage unit 106 may include one or more databases. In certain embodiments, a portion of all of storage unit 106 may include a database, such as one or more structured query language (SQL) servers or relational databases. In certain embodiments, a portion or all of storage unit 106 may implement a portion or all of a blockchain. The one or more storage units 106 may be implemented using one or more computer systems/servers and may be connected to a network via which user devices may be able to access/update associated data records.

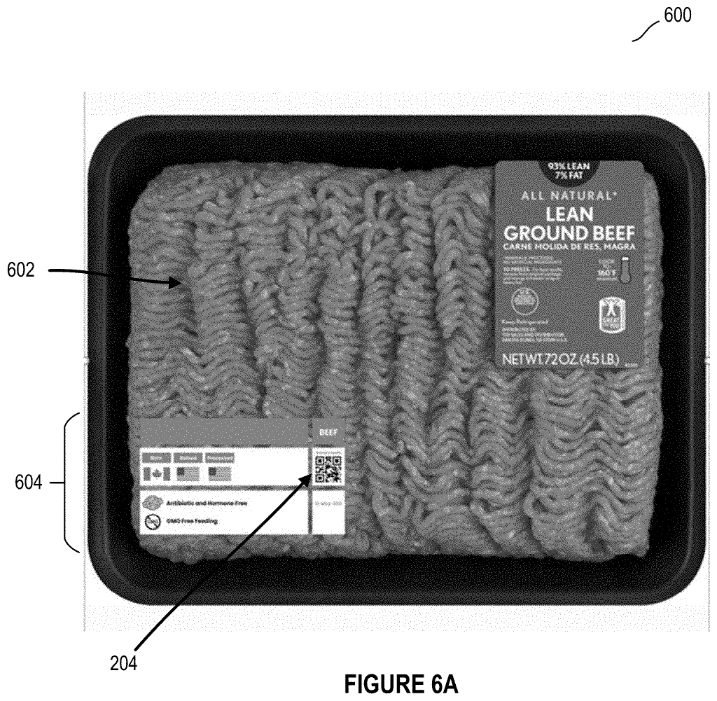

Storage unit 106 may be used to store digital animal records 110 that contain information that facilitates managing animal health of a plurality of animals. In certain embodiments, digital animal records 110 may include animal information, which may include animal inventory information, animal health information, any combination of the preceding, or other information that may facilitate managing animal health of a plurality of animals. Additional detail regarding digital animal records 110 is described below with reference to FIG. 5.

As just one example, embodiments may store collected information on one or more networked storage devices (e.g., in the so-called cloud). Certain embodiments use human-grade Health Insurance Portability and Accountability Act of 1996 (HIPAA)-compliant security for privacy. In certain embodiments, users associated with a farm 102a may be able to update/access at least some of the same data records as stored on the network so that such users are viewing or otherwise interacting with synchronized data.

As described in greater detail below, users associated with entities 102 may communicate with processing system 104 to, among other tasks, request one or more codes for identifying a source animal and/or an animal-based meat product derived from a source animal, and/or to request information based on a scan of the one or more codes.

Entities 102 may communicate with processing system 104, and potential with each other, via network 112. Network 112 may include wireless and/or wireline platforms operable to exchange electronic data or information. In certain embodiments, network 112 may include one or more packet data networks offering communication interfaces or exchanges between components of system 100. Network 112 include any suitable combination of local area networks (LANs), metropolitan area networks (MANs), wide area networks (WANs), wireless local area networks (WLANs), virtual private networks (VPNs), intranets, Internets, plain old telephone systems (POTSs), or any other appropriate architecture or system that facilitates electronic communications. Certain networks 112 include wireless technology such as WiMax (802.16), WiFi (802.11), 3G, 4G, 5G, or any other suitable wireless technologies in any suitable combination.

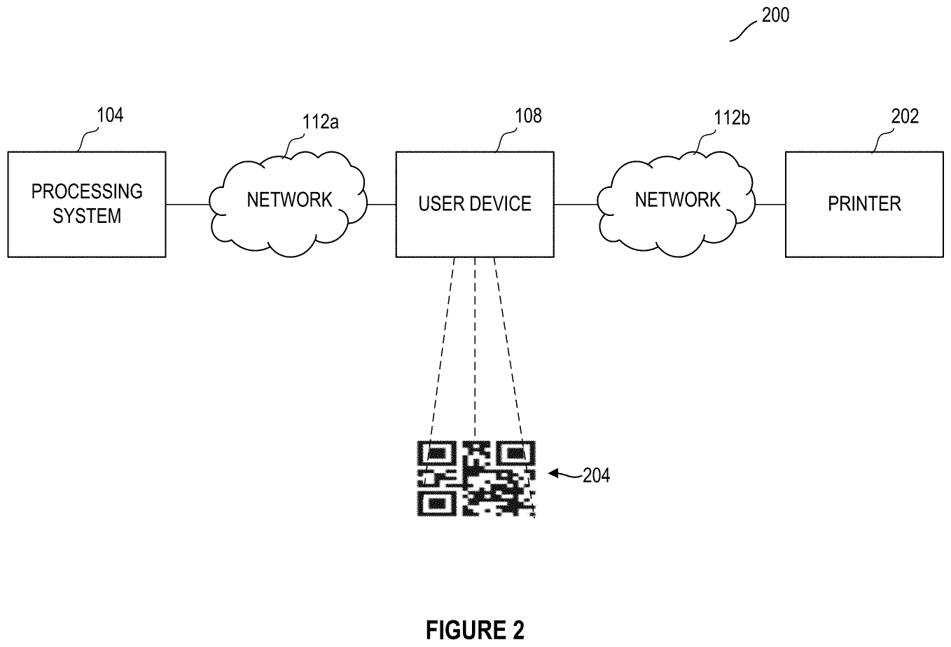

FIG. 2 illustrates an example system 200 for facilitating the traceability of animal-based meat products, according to certain embodiments. In certain embodiments, obtaining one or more codes (e.g., machine-readable image files and/or physical representations of machine-readable image files) for a source animal of one or more animal-based meat products and/or obtaining animal information (e.g., location information or health information associated with a source animal of one or more animal-based meat products) for a source animal of one or more animal-based meat products using a code.

System 200 generally includes one or more processing systems 104 operable to communicate with one or more user devices 108 through one or more networks 112a, each referred to hereinafter in the singular for simplicity. In certain embodiments, user device 108 may also be operable to communicate using one or more networks 112b to a printer 202.

As described further below, an entity 102 may desire to obtain a code 204 for a source animal of an animal-based meat product and/or obtain, using code 204, animal information (e.g., location information or health information) for a source animal associated with an animal-based meat product. In certain embodiments, code 204 may make certain information associated with a source animal for one or more animal-based meat products accessible to a user of user device 108. For example, code 204 may be provided with the animal-based meat product and may be used to convey information.

User device 108 may be used to optically image or scan code 204 and decode information conveyed by code 204. The information conveyed by code 204 and decoded by user device 108 may be used, in certain instances, to obtain and print a physical representation of code 204 to label a source animal or an animal-based meat product (e.g., a carcass or a sub-product derived from the carcass) derived from a source animal for tracking the animal-based meat product to the source animal. For example, digital animal records 110 may be updated with the code 204, so that animal-based meat products may be tracked to the source animal using code 204. The information conveyed by code 204 and decoded by user device 108 may be used, in certain instances, to obtain and print a physical representation of code 204 to label an animal-based meat product (e.g., a carcass or a sub-product derived from the carcass) derived from a source animal for placement on a label that is made available to a purchaser 102d of the animal-based meat product for the purchaser 102d to obtain certain animal information about the source animal associated with the animal-based meat product. The information conveyed by code 204 and decoded by user device 108 may be used, in certain instances, to obtain certain animal information about the source animal associated with the animal-based meat product.

The descriptions of processing system 104 and user device 108 from FIG. 1 are incorporated by reference. Additionally, networks 112a and 112b may be all or a part of network 112 described above with reference to FIG. 1, and the description of network 112 from FIG. 1 is incorporated by reference. Although FIG. 2 illustrates networks 112 and 112b as being separate from each other, in certain embodiments networks 112a and 112b may be included in the same network 112.

User device 108 generally refers to any suitable computing device(s) capable of requesting generation of a code 204 and/or using code 204 to access animal information associated with a source animal of an animal-based meat product. For example, user device 108 may include a digital camera and an application for decoding the information conveyed by code 204. User device 108 may use the information decoded from code 204 to transmit a request (e.g., for receipt by processing system 104) for accessing animal information associated with a source animal of an animal-based meat product.

Code 204 may convey information that facilitates accessing animal information associated with a source animal of an animal-based meat product. For example, the animal information may include location information for the source animal of an animal-based meat product, such as the farm 102a at which the source animal was raised, the meat processing plant 102b at which the source animal (e.g., an animal carcass of the source animal) was processed, the butcher 102c at which the animal carcass was processed into subparts for sale, or the like. As another example, the animal information may include health information for the source animal of an animal-based meat product. In certain embodiments, the additional information may be encoded in code 204 (e.g., a physical representation of a machine-readable image), or code 204 may direct the user device 108 to a website that provides the additional information to the user based on identifying information (e.g., for the source animal) included in a request generated from a scan of the code 204.

For example, code 204 may convey any suitable combination of text-based instructions and/or queries, logic for accessing a webpage (e.g., a Uniform Resource Location, a Uniform Resource Identifier, IP address, etc.), information regarding the animal-based meat product and/or the source animal of the animal-based meat product, an identification of the animal-based meat product and/or the source animal of the animal-based meat product, and other information that facilitates obtaining information regarding the source animal of an animal-based meat product.

Code 204 may convey other information as a machine-readable pattern of geometric shapes, colors, and/or spacing between geometric shapes. For example, code 204 may be a printed label having one or more one-dimensional barcodes, one or more quick resource codes (QR codes), one or more multidimensional barcodes, a combination of a two-dimensional dot matrix code and a single-dimensional barcode, or any of a variety of other patterns of shapes, geometric shapes, colors, and/or spacing between geometric shapes, or any combination of the foregoing. Although several example codes 204 are described, any suitable code 204 may be used. In certain instances, code 204 may be attached, printed, or otherwise integrated into packaging of an animal-based meat product, a tag on a source animal or on an animal-based meat product, such that code 204 may be visible to users associated with entities 102, such as purchasers 102d.

In certain instances, an animal-based meat product refers to any meat product sourced from a source animal at any stage from slaughtering of the source animal to consumption as a food item. For example, an animal-based meat product may be an animal carcass following slaughter of a source animal. As another example, an animal-based meat product may be a subpart of a source animal cut from the animal carcass.

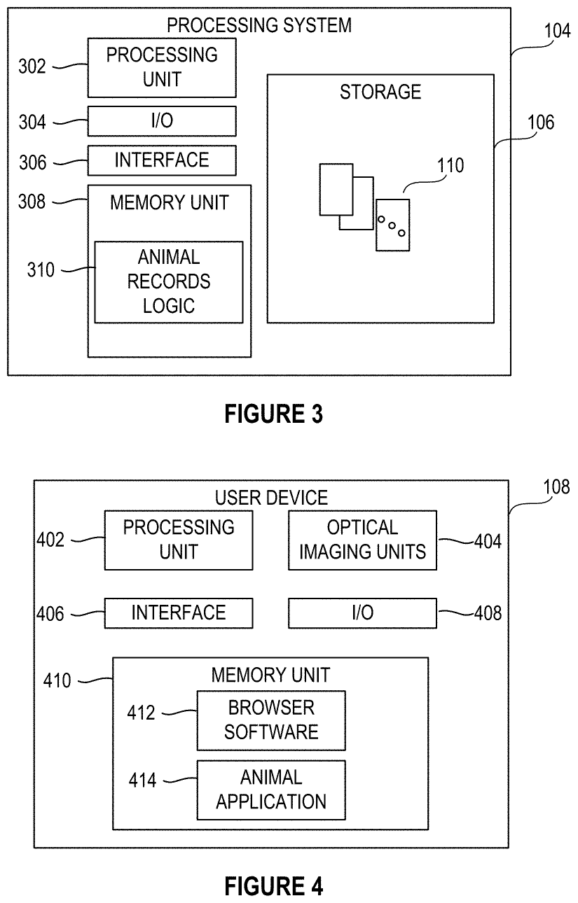

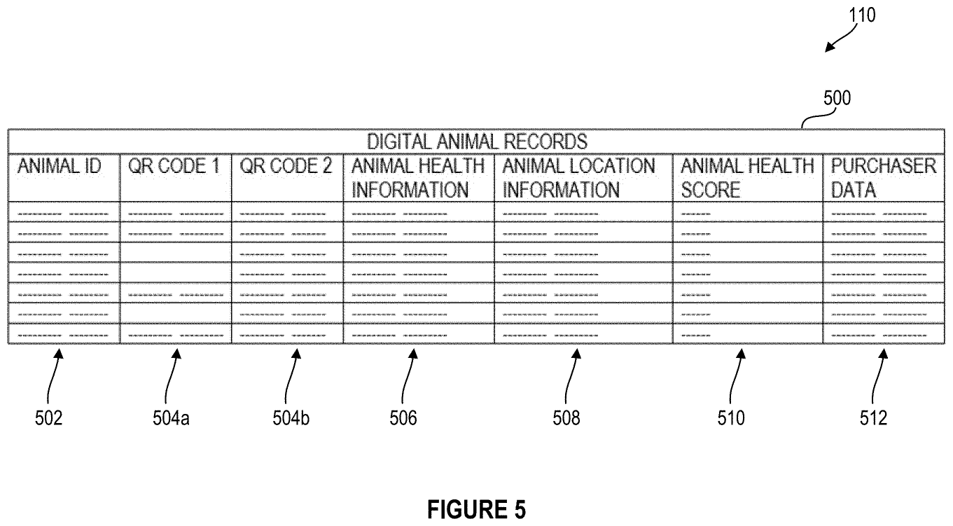

Example operation of processing system 104 and user devices 102 are described in greater detail below with reference to FIGS. 3 and 4, respectively.

While FIGS. 1 and 2 illustrate various components, in certain alternative embodiments some or all of the functionality of the present disclosure may be implemented using memory and/or processors of one or more other devices in communication with the illustrated components. In addition, some or all of the components and/or data structures of system 100 may also be stored (e.g., as software instructions or structured data) on a computer-readable medium, such as one or more of a hard disk, a memory, a network, and/or a portable media article (e.g., a DVD or flash memory devices) to be read by an appropriate drive or via an appropriate connection. The system 100 components and data structures may also be transmitted as generated data signals (e.g., by being encoded in a carrier wave or otherwise included as part of an analog or digital propagated signal) on a variety of computer-readable transmission mediums, including wireless-based and wired/cable-based mediums, and can take a variety of forms (e.g., as part of a single or multiplexed analog signal, or as multiple discrete digital packets or frames). Such computer program products may also take other forms in other embodiments. Accordingly, the described functionality may be implemented using other configurations.

FIG. 3 illustrates additional details of the example processing system 104 and storage unit 106, according to certain embodiments. In the illustrated embodiment, processing system 104 includes one or more processing units 302, various I/O functionality 304, one or more interfaces 306, animal records logic 310 encoded in memory unit 308, and storage unit 106, each referred to hereinafter in the singular for simplicity. In certain embodiments, processing system 104 includes a combination of hardware, firmware, and software that facilitates generating codes 204, providing animal information in response to a request resulting from a scan (e.g., by a user device 108) of a code 204, or other suitable operations.

Processing unit 302 may refer, for example, to the one or more tangible devices of processing system 104 capable of carrying out instructions, such as that of a computer program. For example, processing unit 302 may include one or more microprocessors, controllers, or any other suitable computing devices or resources. Processing unit 302 may work, either alone or with other components of processing system 104, to provide a portion or all of the functionality of processing system 104 described herein. For example, processing unit 302 may be execute or realize various logic-based functions of animal records logic 310.

I/O functionality 304 may include, for example, any communication method of processing system 104 (e.g., from an image buffer to a display). Input functionality may include, for example, the receiving signals or data by processing system 104. Output functionality may include, for example, the communication of signals or data by processing system 104. These terms may also refer to part of an action, such as, for example, to “perform I/O” may involve performing an input and/or output operation. In some cases, one or more communication devices of processing system 104 may be used to implement both input and output functionality.

Interface 306 may include, for example, any tangible device(s) that may be used (e.g., by a person, or by another device or system) to communicate with processing system 104. In certain embodiments, any suitable combination of keyboards, mice, touch screens, and speech recognition devices may be considered an input interfaces 306 and monitors and printers may be considered output interfaces 306.

Memory unit 308 may include any number of tangible storage media. Additionally, all or part of memory unit 308 could reside locally within processing system 104 or could reside at a location external but accessible to processing system 104 (e.g., within removable media, and/or at an external database). In certain embodiments, memory unit 308 may take the form of a suitable combination of volatile and non-volatile memory including, without limitation, magnetic media, optical media, RAM, ROM, removable media, or any other suitable memory component. Where appropriate, RAM may be DRAM or SRAM. Moreover, where appropriate, this RAM may be single-ported or multi-ported RAM. This disclosure contemplates any suitable RAM.

The description of storage unit 106 from FIG. 1 is incorporated by reference. Additionally, all or part of storage unit 106 could reside locally within processing system 104 or could reside at a location external but accessible to processing system 104 (e.g., within removable media, and/or at an external database).

As described above, storage unit 106 may store digital animal records 110 that include animal information regarding source animals. In certain embodiments, digital animal records 110 may include animal information, which may include animal inventory information, animal health information, any combination of the preceding, or other information that may facilitate managing animal health of a plurality of animals. For example, the animal inventory information may include various biographical information about the source animals, such as an identifier, a sex, a birthdate, location information, or any other suitable information that may be useful in maintaining an inventory of source animals. As another example, health information about the source animals may include medical records, vaccination dates, health events, treatments, breeding records, or any other suitable health information. Additional detail regarding example digital animal records 110 are described below with reference to FIG. 5.

In certain embodiments, the described processing and memory elements (such as memory unit 308 and processing unit 302) may be distributed across multiple devices such that the operations performed utilizing these elements may also be distributed across multiple devices. For example, software operated utilizing these elements may be run across multiple computers that contain these processing and memory elements. Other variations aside from the stated example are contemplated involving the use of distributed computing.

In this example, animal records logic 310 is stored within memory unit 308; however, animal records logic 310 may be stored external to memory unit 308 and/or external to processing system 104. Animal records logic 310 is an application that facilitates generating a code 204 for a source animal of an animal-based meat product, updating digital animal records 110 with generated codes 204, obtaining (e.g., in response to a request from a user device 108 having scanned a code 204) animal information (e.g., location information or health information) for a source animal associated with an animal-based meat product according to code 204, and other suitable functionality.

In certain embodiments, animal records logic 310 is configured to generate, according to the animal information of digital animal records 110, one or more animal health scores for the source animals, and to update digital animal records 110 with the animal health score. In certain embodiments, the meat facts that are made available via codes 204 may include the one or more animal health scores. In certain embodiments, at least a portion of animal records logic 310 is implemented using an artificial intelligence (AI) engine that is able to analyze digital animal records 110 and determine the one or more animal health scores. As just one particular example, in certain embodiments, at least a portion of animal records logic may be implemented using a FUSION AI engine.

In operation of an example embodiment of processing system 104, processing system 104 may receive a request to generate a first code 204 for identifying an animal source associated with an animal-based meat product. For example, processing system 104 may receive the request from a user device 108 of a user associated with an entity 102. As a first particular example, the request may be received from a user of a user device 108a at farm 102a. As a second particular example, the request may be received from a user of a user device 108b at animal processing plant 102b. In one example scenario, once source animals are inspected by an appropriate authority (e.g., the United States Department of Agriculture Food Safety and Inspection Service), line workers may identify the animal identifier with a code 204 (e.g., a QR code) generated at the feedlot and generate a new carcass code 204 (e.g., a QR code) label for the carcass. In certain embodiments, source animals culled by an inspector at antemortem or postmortem time periods may be voided from the active labeling database of storage system 104. Carcasses of source animals with the generated code 204 may be shipped to butcher 102c for custom butchering. As a third particular example, the request may be received from a user of a user device 108c at butcher 102c.

Processing system 104 may access, from digital animal records 110, a digital animal record 110 corresponding to the source animal associated with the animal-based meat product. Processing system 104 may generate, based on the digital animal record 110 corresponding to the source animal associated with the animal-based meat product, a first code 204 for identifying the source animal. Processing system 104 may update the digital animal record 110 corresponding to the source animal associated with the animal-based meat product with the first code 204 for identifying the source animal. Processing system 104 may transmit the first code 204 to a receiving device. Although the receiving device could be any device, in certain embodiments, the receiving device is the user device 108 that requested the first code 204.

Processing system 104 may receive a request to generate a second code 208 for a packaging label associated with the animal-based meat product. For example, processing system 104 may receive the request from a user device 108 of a user associated with an entity 102. As a particular example, the request may be received from a user of a user device 108c at butcher 102c. In one example scenario, a grocery store meat department may receive source animal carcasses with the generated code 204, and each package of meat cut from the carcass may include a meat label with a requested code 204 with identifying data.

Processing system 104 may access, from digital animal records 110, a digital animal record 110 corresponding to the source animal associated with the animal-based meat product. Processing system 104 may generate, based on the digital animal record 110 corresponding to the source animal associated with the animal-based meat product, a second code 204 for accessing health information associated with the source animal of the animal-based meat product. Processing system 104 may update the digital animal record 110 corresponding to the source animal associated with the animal-based meat product with the second code 204. Processing system 104 may transmit the second code 204 to a receiving device. Although the receiving device could be any device, in certain embodiments, the receiving device is the user device 108 that requested the second code 204.

In operation of another example embodiment of processing system 104, processing system 104 may receive a label-generating request for generating a machine-readable image file for an animal-based meat product, the animal-based meat product corresponding to a particular source animal. For example, processing system 104 may receive the request from a user device 108 of a user associated with an entity 102. As a first particular example, the request may be received from a user of a user device 108a at farm 102a. As a second particular example, the request may be received from a user of a user device 108b at animal processing plant 102b. As a third particular example, the request may be received from a user of a user device 108c at butcher 102c. For example, a person associated with farm 102a may wish to obtain a code 204 for attaching to a source animal being raised on farm 102a. As another example, a person associated with meat processing plant 102b may wish to obtain a code 204 for attaching to a carcass of a source animal. As another example, a person associated with butcher 102c may wish to obtain a code 204 for attaching to one or more animal-based meat products (e.g., as part of a label for affixing on packaging).

Although the particular source animal could be any type of animal that may be a source of an animal-based meat product, in certain embodiments, the particular source animal is a cow or a pig. For example, the animal-based meat product may be an animal carcass from which multiple sub-products are to be formed. As another example, the animal-based meat product may be a meat sub-product formed from an animal carcass.

Processing system 104 may access, in response to receiving the label-generating request, digital animal records 110 for source animals to identify a digital animal record 110 for the particular source animal corresponding to the animal-based meat product. The digital animal records 110 may include animal information for multiple source animals, and the animal information may include identification information, location information, and health information for the source animals.

Processing system may generate, based on the animal information of the digital animal record 110 for the particular source animal of the animal-based meat product, a machine-readable image file (e.g., a code 204) for the particular source animal of the animal-based meat product. The machine-readable image file, when decoded, may make available meat facts regarding the particular source animal of the animal-based meat product.

In certain embodiments, processing system 104 may encode machine-readable data into a plurality of image components. The encoded data may convey information that obtaining animal information (e.g., meat facts, such as animal location information, animal health information, or other suitable information) about the source animal of an animal-based meat product. For example, the data may convey any suitable combination of the animal information itself (e.g., the meat facts themselves), queries, logic for accessing a webpage (e.g., a Uniform Resource Location, a Uniform Resource Identifier, IP address, etc.), information regarding a purchaser of the animal-based meat product, or other suitable information. In certain embodiments, the data encoded in code 204 may convey information that facilitates communication between user device 108 and processing system 104.

Processing system 104 may generate an image file that facilitates obtaining the animal information. For example, the data encoded as a plurality of image components may be used to generate an image file that includes a machine-readable pattern of those image components. The pattern of image components may include, for example, geometric shapes, colors, and/or spacing between geometric shapes, which collectively represent the encoded data. In certain embodiments, the image file may include one or more barcodes, QR codes, other code types, and/or any suitable combination thereof. Particular image files include content that may be printed or otherwise displayed on tangible media as codes 204.

Processing system 104 may update the digital animal records 110 with the machine-readable image file. Processing system 104 may transmit the machine-readable image file to a requesting device. In certain embodiments, the machine-readable image file that is transmitted to the requesting device is a physical representation of the machine-readable image file that is printable, and the physical representation of the machine-readable image file includes one or more of a QR code or a barcode. Although the requesting device could be any device, in certain embodiments, the requesting device is the user device 108 that submitted the label-generating request.

Processing system 104 may receive an information request from a user device 108 that has decoded the website address from an optical scan of a package label that includes a physical representation of the machine-readable image file. The information request may provide the animal identifier corresponding to the particular source animal. For example, a purchaser 102d or another user may wish to obtain animal information (e.g., meat facts) associated with one or more animal-based meat products.

Processing system 104 may access, according to the animal identifier and from the digital animal records 110 for the source animals, the health information for the particular source animal. Processing system 104 may generate, according to the health information for the particular source animal, data for receipt by the user device, the data comprising the meat facts regarding the particular source animal. Processing system may transmit the data to the user device 108 for display.

For example, in certain embodiments, the machine-readable image file, when decoded, includes a website address to a website that provides the meat facts about the particular source animal of the animal-based meat product based at least in part on an animal identifier. As another example, in certain embodiments, the machine-readable image file, when decoded, includes the meat facts about the particular source animal of the animal-based meat product.

Regardless of how they are accessed, in certain embodiments, the meat facts include one or more of information regarding vaccinations of the particular source animal, information regarding antibiotics of the particular source animal, information regarding respiratory health of the particular source animal, information regarding liver health of the particular source animal, information regarding neurological health of the particular source animal, and/or any other suitable information. In certain embodiments, processing system 104 may generate, according to the animal information, an animal health score for the particular source animal, and may update the digital animal records 110 with the animal health score. The meat facts may include the animal health score.

Processing system 104 may receive, after a sale of the animal-based meat product, purchaser data. The purchaser data may include purchaser-identifying information and a date of purchase. Processing system 104 may update the digital animal records with the purchaser data so that a sale of the animal-based meat product is linked to a particular purchaser.

In certain embodiments, processing system 104 may receive a request to determine location information associated with the particular source animal for animal-based meat product, and the request may include a purchaser identifier for a purchaser of the animal-based meat product. Processing system 104 may access, according to the purchaser identifier and the purchaser-identifying information of the purchaser data, the digital animal record 110 for the particular source animal of the animal-based meat product to determine location information for the particular source animal, and may transmit the location information for the particular source animal.

FIG. 4 illustrates additional details of the example user device 108, according to certain embodiments. As shown in FIG. 4, user device 108 may include one or more processing units 402, one or more optical imaging units 404, one or more interfaces 406, various I/O functionality 408, and one or more memory units 410, each referred to hereinafter in the singular for simplicity. In certain embodiments, user device 108 includes a combination of hardware, firmware, and software that facilitates imaging and decoding codes 204.

Processing unit 402 may refer, for example, to the one or more tangible devices of user device 108 capable of carrying out instructions, such as that of a computer program. For example, processing unit 402 may include one or more microprocessors, controllers, or any other suitable computing devices or resources. Processing unit 402 may work, either alone or with other components of user device 108, to provide a portion or all of the functionality of user device 108 described herein. For example, processing unit 402 may be execute or realize various logic-based functions of browser software 412 and/or animal application 414.

Each optical imaging unit 404 may be configured to generate electronic data representative of an optical scan of code 204. For example, optical imaging unit 404 may include a digital camera capable of generating digital photographs of code 204. As another example, imaging unit 404 may include a scanner (e.g., a barcode scanner) capable of translating optical impulses into electrical ones.

Interface 406 may include, for example, any tangible device(s) that may be used (e.g., by a person, or by another device or system) to communicate with user device 108. For example, any suitable combination of keypads, touch screens, and speech recognition devices may be considered input interfaces 406 and a display screen may be considered output interfaces 406.

I/O functionality 408 may include, for example, any communication method of user device 108 (e.g., from an image buffer to a display). Input functionality may include, for example, the receiving signals or data by user device 108. Output functionality may include, for example, the communication of signals or data by user device 108. These terms may also refer to part of an action, such as, for example, to “perform I/O” may involve performing an input and/or output operation. In some cases, one or more communication devices of user device 108 may be used to implement both input and output functionality.

Memory unit 410 may include any number of tangible storage media. Additionally, all or part of memory unit 410 could reside locally within user device 108 or could reside at a location external but accessible to user device 108 (e.g., within removable media, and/or at an external database). In certain embodiments, memory unit 410 may take the form of a suitable combination of volatile and non-volatile memory including, without limitation, magnetic media, optical media, RAM, ROM, removable media, or any other suitable memory component. Where appropriate, RAM may be DRAM or SRAM. Moreover, where appropriate, this RAM may be single-ported or multi-ported RAM. This disclosure contemplates any suitable RAM. Memory unite 410 may include, for example, applications programs, network, communication programs, operating system software data, etc. In certain embodiments, memory unit 410 may include decoder software capable of analyzing image data provided by optical imaging unit and decoding the information conveyed by code 204.

User device 108 may include browser software 412, which in this example is stored within memory unit 410. Browser software 412 generally refers to any suitable software application that enables a user to display and interact with information (e.g., text, images, videos, music, etc.) typically located on a web page at a web site (e.g., a web site on the World Wide Web, a local area network, hosted by processing system 104, etc.).

User device 108 may include, or may be configured to access, animal application 414 that when executed facilitates requesting a code 204 for a source animal associated with one or more animal-based meat products and/or accessing animal information using information obtained from a scan of code 204. In certain embodiments (e.g., for a user device 108 of a user associated with farm 102a), animal application 414 may facilitate providing animal information to processing system 104 for storage as digital animal records 110. Animal application 414 may be implemented in any suitable combination of hardware, firmware, and software. In certain embodiments, animal application 414 includes a set of computer-readable instructions (e.g., stored in memory unit 410 or some other suitable computer-readable storage medium) that when executed by processing unit 402 are operable to perform certain operations. For example, animal application 414 may use the information decoded from code 204 to execute a program and/or generate a request that initiates a procedure for requesting generation of a code 204 and/or accessing animal information in response to a scan of code 204.

In operation of an example embodiment of user device 108, user device 108 may decode data coded in an optical scan of a physical representation of a machine-readable image file (e.g., a code 204) located on a label for an animal-based meat product. For example, a purchaser 102d or another user may wish to obtain animal information (e.g., meat facts) associated with one or more animal-based meat products. The user may use user device 108 to optically image or scan a code 204 that may be provided in connection with one or more animal-based meat products. The user device 108 may also decode information conveyed by the optical image or scan of code 204. User device 108 may present some or all of the decoded information to the person (e.g., using a display and/or speakers of user device 108).

User device 108 may access, according to the data coded in the optical scan, meat facts about a particular source animal corresponding to the animal-based meat product. In certain embodiments, the physical representation of the machine-readable image file includes one or more of a QR code or a barcode, and the data coded in the optical scan is coded in the QR code or barcode and includes the meat facts. The data coded in the optical scan of the label may include the meat facts about the source animal associated with the meat product, and accessing, by the user device 108 according to the data coded in the optical scan, the meat facts about the particular source animal corresponding to the animal-based meat product may include determining the meat facts directly from the data coded in the optical scan of the physical representation of the machine-readable image file.

In certain embodiments, the meat facts are determined by a processing system (e.g., processing system 104) distinct from the user device 108. The processing system (e.g., processing system 104) may have access to digital animal records 110 for multiple source animals that include the particular source animal such that the digital animal records 110 includes a particular data record corresponding to the particular source animal. In certain embodiments, the processing system 104 may have generated the machine-readable image file according to the particular data record corresponding to the particular source animal.

In certain embodiments, the data coded in the optical scan of the label includes an identifier for mapping to a digital animal record 110 for the particular source animal for the animal-based meat product and a website address (e.g., a URL). In such an embodiments, accessing, by the user device 108 according to the data coded in the optical scan, the meat facts about the particular source animal corresponding to the animal-based meat product may include initiating transmission of a request using the website address, the request comprising the identifier, and receiving, in response to the request, the meat facts about the particular source animal corresponding to the animal-based meat product. In certain embodiments, the meat facts are received from a processing system (e.g., processing system 104) distinct from the user device 108, the processing system having determined the meat facts by accessing digital animal records 110 for a plurality of source animals and determining the meat facts for the particular source animal according to the identifier included in the request. In certain embodiments, the meat facts include health information associated with the particular source animal.

User device 108 may display the meat facts about the particular source animal associated with the animal-based meat product.

FIG. 5 illustrates additional details of example digital animal records 110, according to certain embodiments. In this example, digital animal records 110 are organized in the form of a table 500; however, any suitable data organization may be used. Table 500 includes a series of rows and columns, with each row corresponding to a respective source animal and each column corresponding to respective animal information regarding the source animals.

For example, column 502 may an animal identifier. The animal identifier could be a series of alphanumeric characters or any other suitable identifier unique to each source animal. In certain scenarios, the animal identifier may be originally assigned to the source animal when the source animal is entered into the system at farm 102a.

As another example, columns 504 (e.g., columns 504a and 504b) represent one or more columns that include a code 204 assigned to the source animal. For example, processing system 104 may generate and assign to a source animal and/or an animal-based meat product a code 204 in response to a request from a user of a user device 108, and processing system 104 may update digital animal records 110 (e.g., table 500) with the generated code 204 in the appropriate row for the appropriate source animal. One or more codes 204 could be generated throughout the path of a source animal from farm 102a, to a carcass at animal processing plant 102b, to butcher 102c, and the like. For certain entries, if a code 204 has not been requested for the source animal (e.g., from a user of a user device 108), then one or more of the code entries may be blank.

As another example, column 506 may include animal health information for each source animal. In certain embodiments, animal health information may include the vaccination record for the source animal, the deworming record for the source animal, the antibiotic record and withdrawal times for the source animal, any respiratory infections (e.g., pneumonia, shipping fever, tuberculosis, etc.) for the source animal, any liver infections (e.g., liver flukes, liver cirrhosis, liver tumors, etc.) for the source animal, neurological diseases (e.g., bovine spongiform encephalopathy (mad cow disease)) for the source animal, and/or any other suitable animal health information.

As another example, column 508 may include animal location information. Animal location information may include a location of the farm 102a at which the source animal was raised, a location of the meat processing plant 102b at which the source animal (e.g., the carcass of the source animal) was processed, a location of the butcher from which animal-based meat products of the source animal were bought, and the like.

As another example, column 510 may include one or more animal health scores. The animal health scores may be based on any suitable information, including any suitable animal information from digital animal records 110. As a particular example, the animal health scores may be based at least in part on the animal health information of column 506. A user of user device 108 may enter animal health scores and/or processing system 104 may generate the one or more health scores. For example, animal records logic 310 may be configured to parse suitable animal information, such as animal health information, and determine one or more animal health scores. In certain embodiments, animal health scores may serve as an abstracted value that can be provided to a user (e.g., a purchaser 102d) to provide animal health information without providing the details of the animal health information. For example, certain categories of animal health information could be rated on a numbered scale (e.g., 1 to 5, with either 1 or 5 being the best, depending on the implementation) to provide an indication of the health rating for that category.

Column 512 may store purchaser data. The purchaser data may be provided by butcher 102c to identify the purchasers of animal-based meat products derived from source animals. For example, an operator of processing system 104 may purchase or otherwise obtain purchaser data from butcher 102c in the form of credit card data, loyalty program data, or the like. In certain embodiments, the purchaser data may include one or more of purchaser identifying information (e.g., a name or other identifier of a purchaser), purchase data information, and any other suitable information. For example, purchaser data may be particularly useful in identifying the source animal associated with a purchaser in the event that a purchaser (or someone linked to a purchaser) contracts a foodborne illness associated with a particular animal-based meat product. This information could then be passed to a public health oversight entity 102e to attempt to locate the source farm 102a, meat processing plant 102b, butcher 102c, or the like to facilitate a recall or other corrective/mitigating action.

Storage unit 106 may be used to store digital animal records 110 (e.g., table 500). For example, processing system 104 may store and update suitable animal information in digital animal records 110 (e.g., table 500) of storage unit 106. The animal information may be provided by one or more of the entities 102 illustrated in FIG. 1 and/or from any other suitable source. Some of the entries in digital animal records 110 (e.g., table 500) may be generated or otherwise provided by processing system 104.

In certain embodiments, users associated with farm 102a may use a livestock animal health management software platform on user devices 102a, which may promote animal health in horses, cattle (e.g., beef or dairy cattle), swine, or other types of animals/livestock. The software platform may be implemented, at least in part, as a software application (e.g., a mobile application or other suitable type of software application) that can run on user devices 108a. At least a portion of the system may be implemented as a mobile application that can be downloaded by a user to a mobile device of the user, such as a mobile smartphone or tablet computer. Users may include any suitable types of users, such as farmers/ranchers and associated users (e.g., employees or otherwise authorized users), or other suitable users. Using the software application, users can add animal information to digital animal records 110 for the source animals associated with farm 102a.

An example implementation of a swine health monitoring application may be a mobile software platform for family and corporate swine operations. The swine platform may include a digital medical record, sick pen inventory, pig location inventories, veterinarian video chat, breeding records, vaccination/dewormer/antibiotic records/reminders. Similar health monitoring applications could be used for cows or other animals that may serve as source animals for animal-based meat products.

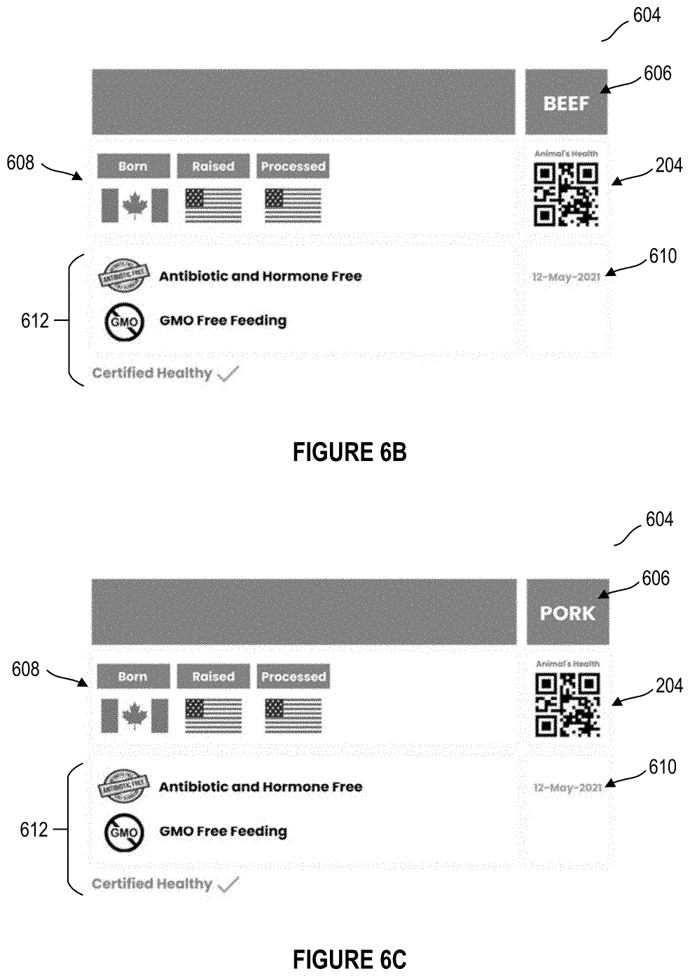

FIGS. 6A-6C illustrate example labels that include a physical representation of a digital image file for a source animal of an animal-based meat product, according to certain embodiments. FIG. 6A illustrates an example meat package 600 of an animal-based meat product 602 and that includes a label 604 having a code 204 printed on the label 604, according to certain embodiments. FIG. 6B illustrates greater detail of an example of label 604 for a beef animal-based meat product 602, according to certain embodiments. FIG. 6C illustrates greater detail of an example of label 604 for a pork animal-based meat product 602, according to certain embodiments.

As shown in FIG. 6A, a label 604 may be affixed to a meat package 600, so that label 604 is visible to a potential purchaser 102d. In the illustrated example, label 604 includes a code 204 that can be scanned (e.g., using a user device 108) by the potential/actual purchaser 102d to learn certain information about the animal-based meat product 602 contained in the package 600. For example, by scanning code 204 using a user device 108, a potential/actual purchaser 102d may learn certain meat facts about the animal-based meat product 602 contained in the package 600. The meat facts may include any suitable animal information, such as any suitable animal inventory information or any suitable animal health information.

As particular examples, the meat facts may include one or more of information regarding vaccinations of the particular source animal, information regarding antibiotics of the particular source animal, information regarding respiratory health of the particular source animal, information regarding liver health of the particular source animal; or information regarding neurological health of the particular source animal. As just one particular example, the meat facts may be presented as the following:

| | |
|---|---|
| Vaccinations | Yes |
| Antibiotics | Yes |
| Respiratory Health | Graded 1-5 (1 being the best) |
| Liver Health | Graded 1-5 (1 being the best) |
| Neurological Health | Graded 1-5 (1 being the best) |

Although certain meat facts are described, this disclosure contemplates the meat facts including any suitable information, according to particular implementations.

As shown in FIG. 6B, label 604 may include a meat type indicator 606, a source indicator 608, a code 204, a date 610, and additional indicators 612. Although meat label 604 is described as including particular information, this disclosure contemplates a meat label including none, some, or all of this information, as well as different information.

Meat type indicator 606 indicates the type of meat according to the type of the source animal of the animal-based meat product 602 included in the package 600 on which the label 604 is affixed. As just a few examples, meat type indicator 606 could indicate pork if the source animal of the animal-based meat product 602 included in package 600 is a pig, beef if the source animal of the animal-based meat product 602 included in package 600 is a cow, chicken if the source animal of the animal-based meat product 602 included in package 600 is a chicken, lamb if the source animal of the animal-based meat product 602 included in package 600 is a sheep, or the like. In the illustrated example of FIG. 6B, meat type indicator 606 indicates that package 600 includes beef.

As described above, label 604 may include a code 204, which may be implemented as a physical representation of a machine-readable image file. Specific examples include a QR code, a barcode, a combination of a QR code and a barcode, or any other suitable type of physical representation of a machine-readable image file. As described above, a scan of code 204 may make available meat facts associated with the animal-based meat product 602 (or the associated source animal) packaged in the package 600 to which label 604 is affixed.

In the illustrated example, source indicator 608 includes three source indicators. A first source indicator indicates where the source animal of the animal-based meat product 602 included in package 600 was born (which might or might not be the same as farm 102a). A second source indicator indicates where the source animal of the animal-based meat product 602 included in package 600 was raised (e.g., farm 102a). A third source indicator indicates where the source animal of the animal-based meat product 602 included in package 600 was processed (e.g., meat processing plant 102b and/or butcher 102c). In this example, source indicators 608 are shown to represent countries but could be more specific, according to particular implementations.

In the illustrated example, date 610 indicates a date in the form of day-month-year. In one example, date 610 indicates the date that the source animal was processed at meat processing plant 102b; however, date 610 could indicate any suitable date (e.g., the date on which the animal-based meat product 602 was packaged into package 600.

In the illustrated example, additional indicators 612 include indicators that the source animal is antibiotic and hormone free, experienced genetically modified organism (GMO)-free feeding, and was certified healthy. In certain embodiments, additional indicators 612 are determined by the entity responsible for operating processing system 104 and/or storage unit 106.

FIG. 6C is generally similar to FIG. 6B, except that label 604 of FIG. 6B is for a pork animal-based meat product rather than a beef animal-based meat product. Thus, the description of FIG. 6B is incorporated by reference.

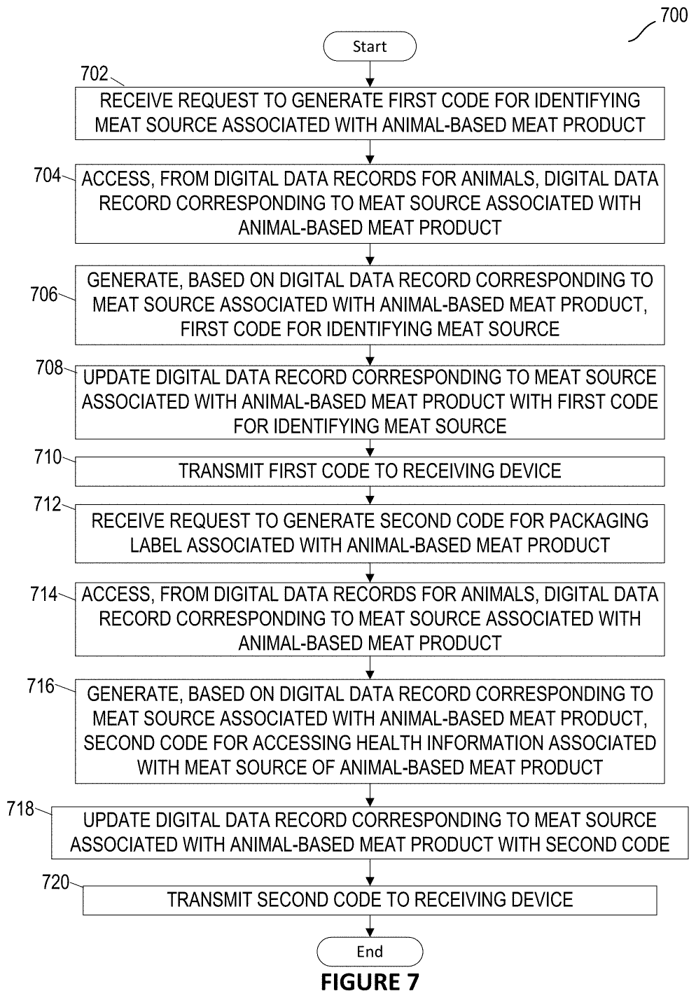

FIG. 7 illustrates an example method 700 for facilitating the traceability of animal-based meat products, according to certain embodiments. For purposes of this example, a storage system (e.g., storage unit 106) stores digital animal records that include identifying information for the plurality of animals and animal health information for the plurality of animals.

At step 702, processing system 104 receives a request to generate a first code 204 for identifying an animal source associated with an animal-based meat product. For example, processing system 104 may receive the request from a user device 108 of a user associated with an entity 102. As a first particular example, the request may be received from a user of a user device 108a at farm 102a. As a second particular example, the request may be received from a user of a user device 108b at animal processing plant 102b. As a third particular example, the request may be received from a user of a user device 108c at butcher 102c.

At step 704, processing system 104 may access, from digital animal records 110, a digital animal record 110 corresponding to the source animal associated with the animal-based meat product.

At step 706, processing system 104 may generate, based on the digital animal record 110 corresponding to the source animal associated with the animal-based meat product, a first code 204 for identifying the source animal.

At step 708, processing system 104 may update the digital animal record 11o corresponding to the source animal associated with the animal-based meat product with the first code 204 for identifying the source animal.

At step 710, processing system 104 may transmit the first code 204 to a receiving device. Although the receiving device could be any device, in certain embodiments, the receiving device is the user device 108 that requested the first code 204.

At step 712, processing system 104 receives a request to generate a second code 208 for a packaging label (e.g., label 604) associated with the animal-based meat product. For example, processing system 104 may receive the request from a user device 108 of a user associated with an entity 102. As a particular example, the request may be received from a user of a user device 108c at butcher 102c.

At step 714, processing system 104 may access, from digital animal records 110, a digital animal record 110 corresponding to the source animal associated with the animal-based meat product.

At step 716, processing system 104 may generate, based on the digital animal record 110 corresponding to the source animal associated with the animal-based meat product, a second code 204 for accessing health information associated with the source animal of the animal-based meat product.

At step 718, processing system 104 may update the digital animal record 11o corresponding to the source animal associated with the animal-based meat product with the second code 204.

At step 720, processing system 104 may transmit the second code 204 to a receiving device. Although the receiving device could be any device, in certain embodiments, the receiving device is the user device 108 that requested the second code 204.

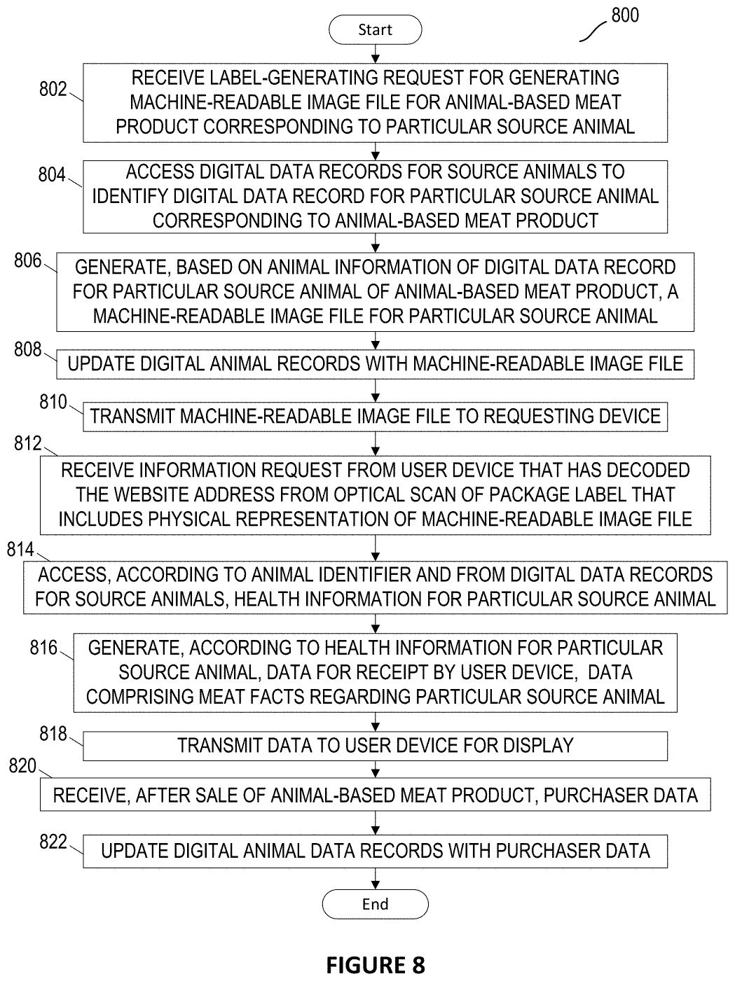

FIG. 8 illustrates an example method 800 for facilitating the traceability of animal-based meat products, according to certain embodiments.

At step 802, processing system 104 receives a label-generating request for generating a machine-readable image file for an animal-based meat product, the animal-based meat product corresponding to a particular source animal. For example, processing system 104 may receive the request from a user device 108 of a user associated with an entity 102. As a first particular example, the request may be received from a user of a user device 108a at farm 102a. As a second particular example, the request may be received from a user of a user device 108b at animal processing plant 102b. As a third particular example, the request may be received from a user of a user device 108c at butcher 102c. For example, a person associated with farm 102a may wish to obtain a code 204 for attaching to a source animal being raised on farm 102a. As another example, a person associated with meat processing plant 102b may wish to obtain a code 204 for attaching to a carcass of a source animal. As another example, a person associated with butcher 102c may wish to obtain a code 204 for attaching to one or more animal-based meat products (e.g., as part of a label 604 for affixing on packaging 600).

Although the particular source animal could be any type of animal that may be a source of an animal-based meat product, in certain embodiments, the particular source animal is a cow or a pig. For example, the animal-based meat product may be an animal carcass from which multiple sub-products are to be formed. As another example, the animal-based meat product may be a meat sub-product formed from an animal carcass.

At step 804, processing system 104 may access, in response to receiving the label-generating request, digital animal records 110 for source animals to identify a digital animal record 110 for the particular source animal corresponding to the animal-based meat product. The digital animal records 110 may include animal information for multiple source animals, and the animal information may include identification information, location information, and health information for the source animals.

At step 806, processing system may generate, based on the animal information of the digital animal record 110 for the particular source animal of the animal-based meat product, a machine-readable image file (e.g., a code 204) for the particular source animal of the animal-based meat product. The machine-readable image file, when decoded, may make available meat facts regarding the particular source animal of the animal-based meat product.

In certain embodiments, processing system 104 may encode machine-readable data into a plurality of image components. The encoded data may convey information that obtaining animal information (e.g., meat facts, such as animal location information, animal health information, or other suitable information) about the source animal of an animal-based meat product. For example, the data may convey any suitable combination of the animal information itself (e.g., the meat facts themselves), queries, logic for accessing a webpage (e.g., a Uniform Resource Location, a Uniform Resource Identifier, IP address, etc.), information regarding a purchaser of the animal-based meat product, or other suitable information. In certain embodiments, the data encoded in code 204 may convey information that facilitates communication between user device 108 and processing system 104.

Processing system 104 may generate an image file that facilitates obtaining the animal information. For example, the data encoded as a plurality of image components may be used to generate an image file that includes a machine-readable pattern of those image components. The pattern of image components may include, for example, geometric shapes, colors, and/or spacing between geometric shapes, which collectively represent the encoded data. In certain embodiments, the image file may include one or more barcodes, QR codes, other code types, and/or any suitable combination thereof. Particular image files include content that may be printed or otherwise displayed on tangible media as codes 204.

At step 808, processing system 104 may update the digital animal records 110 with the machine-readable image file.

At step 810, processing system 104 may transmit the machine-readable image file to a requesting device. In certain embodiments, the machine-readable image file that is transmitted to the requesting device is a physical representation of the machine-readable image file that is printable, and the physical representation of the machine-readable image file includes one or more of a QR code or a barcode. Although the requesting device could be any device, in certain embodiments, the requesting device is the user device 108 that submitted the label-generating request.

At step 812, processing system 104 may receive an information request from a user device 108 that has decoded the website address from an optical scan of a package label (e.g., label 604) that includes a physical representation of the machine-readable image file. The information request may provide the animal identifier corresponding to the particular source animal. For example, a purchaser 102d or another user may wish to obtain animal information (e.g., meat facts) associated with one or more animal-based meat products.

At step 814, processing system 104 may access, according to the animal identifier and from the digital animal records 110 for the source animals, the health information for the particular source animal.

At step 816, processing system 104 may generate, according to the health information for the particular source animal, data for receipt by the user device, the data comprising the meat facts regarding the particular source animal.

At step 818, processing system may transmit the data to the user device 108 for display.

For example, in relation to steps 812-818, in certain embodiments, the machine-readable image file, when decoded, includes a website address to a website that provides the meat facts about the particular source animal of the animal-based meat product based at least in part on an animal identifier.

As another example, as a potential alternative to steps 812-818 in certain embodiments, the machine-readable image file, when decoded, includes the meat facts about the particular source animal of the animal-based meat product.

Regardless of how they are accessed, in certain embodiments, the meat facts include one or more of information regarding vaccinations of the particular source animal, information regarding antibiotics of the particular source animal, information regarding respiratory health of the particular source animal, information regarding liver health of the particular source animal, information regarding neurological health of the particular source animal, and/or any other suitable information. In certain embodiments, processing system 104 may generate, according to the animal information, an animal health score for the particular source animal, and may update the digital animal records 110 with the animal health score. The meat facts may include the animal health score.

At step 820, processing system 104 receives, after a sale of the animal-based meat product, purchaser data. The purchaser data may include purchaser-identifying information and a date of purchase.

At step 822, processing system 104 may update the digital animal records with the purchaser data so that a sale of the animal-based meat product is linked to a particular purchaser.

In certain embodiments, processing system 104 may receive a request to determine location information associated with the particular source animal for animal-based meat product, and the request may include a purchaser identifier for a purchaser of the animal-based meat product. Processing system 104 may access, according to the purchaser identifier and the purchaser-identifying information of the purchaser data, the digital animal record 110 for the particular source animal of the animal-based meat product to determine location information for the particular source animal, and may transmit the location information for the particular source animal.

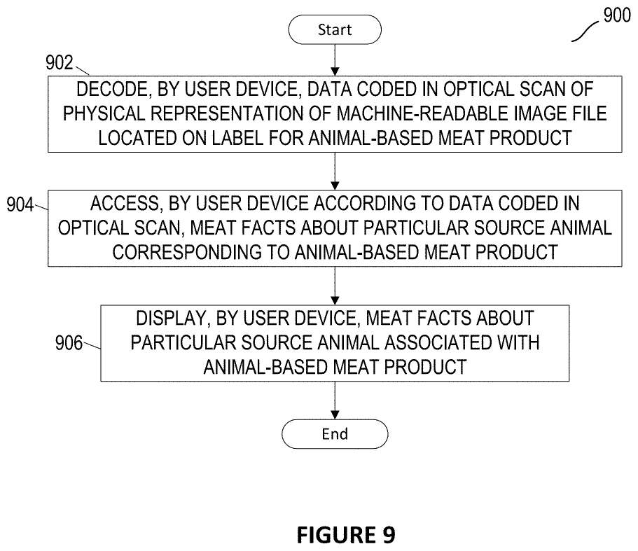

FIG. 9 illustrates an example method 900 for facilitating the traceability of animal-based meat products, according to certain embodiments.

At step 902, a user device 108 may decode data coded in an optical scan of a physical representation of a machine-readable image file (e.g., a code 204) located on a label (e.g., label 604) for an animal-based meat product. For example, a purchaser 102d or another user may wish to obtain animal information (e.g., meat facts) associated with one or more animal-based meat products. The user may use user device 108 to optically image or scan a code 204 that may be provided in connection with one or more animal-based meat products. The user device 108 may also decode information conveyed by the optical image or scan of code 204. User device 108 may present some or all of the decoded information to the person (e.g., using a display and/or speakers of user device 108).

At step 904, user device 108 may access, according to the data coded in the optical scan, meat facts about a particular source animal corresponding to the animal-based meat product.

In certain embodiments, the physical representation of the machine-readable image file includes one or more of a QR code or a barcode, and the data coded in the optical scan is coded in the QR code or barcode and includes the meat facts. The data coded in the optical scan of the label may include the meat facts about the source animal associated with the meat product, and accessing, by the user device 108 according to the data coded in the optical scan, the meat facts about the particular source animal corresponding to the animal-based meat product may include determining the meat facts directly from the data coded in the optical scan of the physical representation of the machine-readable image file.

In certain embodiments, the meat facts are determined by a processing system (e.g., processing system 104) distinct from the user device 108. The processing system (e.g., processing system 104) may have access to digital animal records 110 for multiple source animals that include the particular source animal such that the digital animal records 110 includes a particular data record corresponding to the particular source animal. In certain embodiments, the processing system 104 may have generated the machine-readable image file according to the particular data record corresponding to the particular source animal.

In certain embodiments, the data coded in the optical scan of the label (e.g., label 604) includes an identifier for mapping to a digital animal record 110 for the particular source animal for the animal-based meat product and a website address (e.g., a URL). In such an embodiments, accessing, by the user device 108 according to the data coded in the optical scan, the meat facts about the particular source animal corresponding to the animal-based meat product may include initiating transmission of a request using the website address, the request comprising the identifier, and receiving, in response to the request, the meat facts about the particular source animal corresponding to the animal-based meat product. In certain embodiments, the meat facts are received from a processing system (e.g., processing system 104) distinct from the user device 108, the processing system having determined the meat facts by accessing digital animal records 110 for a plurality of source animals and determining the meat facts for the particular source animal according to the identifier included in the request.

In certain embodiments, the meat facts include health information associated with the particular source animal.

At step 906, the user device 108 may display the meat facts about the particular source animal associated with the animal-based meat product.

Although the present disclosure describes or illustrates particular operations as occurring in a particular order, the present disclosure contemplates any suitable operations occurring in any suitable order. Moreover, the present disclosure contemplates any suitable operations being repeated one or more times in any suitable order. Although the present disclosure describes or illustrates particular operations as occurring in sequence, the present disclosure contemplates any suitable operations occurring at substantially the same time, where appropriate. Any suitable operation or sequence of operations described or illustrated herein may be interrupted, suspended, or otherwise controlled by another process, such as an operating system or kernel, where appropriate. The acts can operate in an operating system environment or as stand-alone routines occupying all or a substantial part of the system processing.

Certain embodiments may provide none, some, or all of the following technical advantages.

Embodiments of this disclosure may implement a traceability capability/program. An example implementation of a food tracking application, according to certain embodiments, may provide an ability to gather and store data for tracking the source of food products, along with (in the case of meat and potentially farm-raised fish) the medical history of the animal from which the meat is sourced and/or the various facts about the farm, and may allow the source of the food and any associated medical or other conditions that may be problematic to be determined or tracked.

Furthermore, certain embodiments include generating a machine-readable image file that, when decoded, comprises a link to or otherwise makes available information determined from digital animal records for an animal. The information may include a source of the animal and/or one or more health facts associated with the animal. A physical representation of the machine-readable image file may include a QR code, a barcode, or another appropriate label that may be used throughout the processing of the animal, from potentially being tagged to the animal while alive, to the carcass, and/or ultimately to the meat packaging. Furthermore, once scanned, the code may cause the device associated with a user (e.g., a meat consumer, a food inspector, the FDA, the CDC, or another suitable user) to access information associated with the history of the animal/farm from which the meat is sourced. While this may be useful for a variety of purposes, including for the general interest of the consumer, in certain embodiments this may be particularly useful in the case of a need to recall food that is sourced from a particular farm or set of farms and/or to determine the source of the need for a recall.

Using one or more codes (e.g., physical representations of machine-readable image files and associated records in digital animal records), certain embodiments may provide an ability to track a source animal from a location at which the source animal was raised to locations at which the source animal is process (e.g., as a carcass) (e.g., at a processing plant) and ultimately formed into one or more animal-based meat products for purchase and consumption (e.g., at a grocer/butcher).

Certain embodiments provide an ability to determine a source of a source animal for an animal-based meat product. For example, the source of the source animal may be the particular farm on which the animal was raise, the processing plant at which the carcass of the source animal was processed, or other suitable locations. In the event that a foodborne illness is contracted by a purchaser/consumer of an animal-based meat product sourced from the source animal, an ability to determine the source animal and one or more locations at which the source animal was raised/processed may facilitated tracking other potentially-affected meat-based food products that may be capable of producing the same or a similar foodborne illness. To the extent appropriate, determining the location may facilitate an accurate, focused food recall, as it may be possible to determine the source animal, and locations to which animal-based meat products formed from the source animal were distributed.

Certain embodiments provide an ability users (e.g., purchasers of animal-based meat products) to quickly access meat facts associated with animal-based meat products. For example, certain embodiments provide techniques for generating a physical representation of a machine-readable image file that can be printed on a label for an animal-based meat product and optically scanned using a user device of the purchaser of the animal-based meat product to access meat facts associated with a source animal of the animal-based meat product.

Due to the organized digital animal records for the source animals and the consistent identification of the source animals, certain embodiments may provide an efficient ability to present meat facts to purchasers of animal-based meat products or other interested parties.

Certain embodiments provide a highly intuitive user interface for accessing meat facts for animal-based meat products as determined from digital animal records maintained by the system.

Certain embodiments may save time by reducing or eliminating hand-written health records through a partial or complete digital solution. Certain embodiments may reduce costs by allowing decision makers to use real-time data to make timely, potentially improved management decisions.

Example embodiments of this disclosure are summarized here. Other embodiments can also be understood from the entirety of the specification as well as the claims filed herein.

Example 1: A non-transitory computer-readable storage medium stores a program for execution by one or more processors. The program includes instructions for receiving a label-generating request for generating a machine-readable image file for an animal-based meat product that corresponds to a particular source animal. The program further includes instructions for accessing, in response to receiving the label-generating request, digital animal records for source animals to identify a digital animal record for the particular source animal. The digital animal records include animal information that includes identification information, location information, and health information for the source animals. The program further includes instructions for generating, based on the animal information of the digital animal record for the particular source animal, a machine-readable image file for the particular source animal that, when decoded, makes available meat facts regarding the particular source animal, and for transmitting the machine-readable image file to a requesting device.

Example 2: The non-transitory computer-readable storage medium of Example 1, where the machine-readable image file, when decoded, includes a website address to a website that provides the meat facts about the particular source animal of the animal-based meat product based at least in part on an animal identifier.

Example 3: The non-transitory computer-readable storage medium of Example 1, where the program further includes instructions for: receiving an information request from a user device that has decoded the website address from an optical scan of a package label that includes a physical representation of the machine-readable image file, the information request providing the animal identifier, the animal identifier corresponding to the particular source animal; accessing, according to the animal identifier and from the digital animal records for the source animals, the health information for the particular source animal; generating, according to the health information for the particular source animal, data for receipt by the user device, the data including the meat facts regarding the particular source animal; and transmitting the data to the user device for display.

Example 4: The non-transitory computer-readable storage medium of any one of Examples 1-3, where the machine-readable image file, when decoded, includes the meat facts about the particular source animal of the animal-based meat product.

Example 5: The non-transitory computer-readable storage medium of any one of Examples 1-4, where the meat facts include one or more of: information regarding vaccinations of the particular source animal; information regarding antibiotics of the particular source animal; information regarding respiratory health of the particular source animal; information regarding liver health of the particular source animal; or information regarding neurological health of the particular source animal.

Example 6: The non-transitory computer-readable storage medium of any one of Examples 1-5, where the program further includes instructions for: receiving, after a sale of the animal-based meat product, purchaser data that includes purchaser-identifying information and a date of purchase; and updating the digital animal records with the purchaser data so that a sale of the animal-based meat product is linked to a particular purchaser.

Example 7: The non-transitory computer-readable storage medium of Example 6, where the program further includes instructions for: receiving a request to determine location information associated with the particular source animal for animal-based meat product, the request including a purchaser identifier for a purchaser of the animal-based meat product; accessing, according to the purchaser identifier and the purchaser-identifying information of the purchaser data, the digital animal record for the particular source animal of the animal-based meat product to determine location information for the particular source animal; and transmitting the location information for the particular source animal.

Example 8: The non-transitory computer-readable storage medium of any one of Examples 1-7, where the program further includes instructions for: generating, according to the animal information, an animal health score for the particular source animal; and updating the digital animal records with the animal health score, the meat facts including the animal health score.

Example 9: The non-transitory computer-readable storage medium of any one of Examples 1-8, where: the animal-based meat product is an animal carcass from which multiple sub-products are to be formed; or the animal-based meat product is a meat sub-product formed from an animal carcass.

Example 10: The non-transitory computer-readable storage medium of any one of Examples 1-9, wherein the particular source animal is a cow or a pig.

Example 11: The non-transitory computer-readable storage medium of any one of Examples 1-10, where: the machine-readable image file that is transmitted to the requesting device is a physical representation of the machine-readable image file that is printable; and the physical representation of the machine-readable image file includes one or more of a quick resource (QR) code or a barcode.

Example 12: A method includes decoding, by a user device, data coded in an optical scan of a physical representation of a machine-readable image file located on a label for an animal-based meat product. The method further includes accessing, by the user device according to the data coded in the optical scan, meat facts about a particular source animal corresponding to the animal-based meat product. The method further includes displaying, by the user device, the meat facts about the particular source animal associated with the animal-based meat product.

Example 13: The method of Example 12, where: the physical representation of the machine-readable image file includes one or more of a quick resource code (QR code) or a barcode, the data coded in the optical scan being coded in the QR code or barcode and including the meat facts; the data coded in the optical scan of the label includes the meat facts about the source animal associated with the meat product; and accessing, by the user device according to the data coded in the optical scan, the meat facts about the particular source animal corresponding to the animal-based meat product includes determining the meat facts directly from the data coded in the optical scan of the physical representation of the machine-readable image file.

Example 14: The method of Example 13, where the meat facts are determined by a processing system distinct from the user device and having access to digital animal records for source animals, the source animals including the particular source animal such that the digital animal records includes a particular data record corresponding to the particular source animal, the processing system having generated the machine-readable image file according to the particular data record corresponding to the particular source animal.

Example 15: The method of any one of Examples 12-14, where: the data coded in the optical scan of the label includes: an identifier for mapping to a digital animal record for the particular source animal for the animal-based meat product; and a website address; and accessing, by the user device according to the data coded in the optical scan, the meat facts about the particular source animal corresponding to the animal-based meat product includes: initiating transmission of a request using the website address, the request including the identifier; and receiving, in response to the request, the meat facts about the particular source animal corresponding to the animal-based meat product.

Example 16: The method of Example 15, where the meat facts are received from a processing system distinct from the user device, the processing system having determined the meat facts by accessing digital animal records for source animals and determining the meat facts for the particular source animal according to the identifier.

Example 17: The method of any one of Examples 15-16, where the website address includes a Uniform Resource Location (URL).

Example 18: The method of any one of Examples 12-17, where the meat facts include health information associated with the particular source animal.

Example 19: A system includes a storage system that stores digital animal health records for animals, the digital animal health records including identifying information and health records for the animals. The system further includes one or more processors and a non-transitory computer-readable storage medium storing a program for execution by the one or more processors. The program includes instructions for receiving a request to generate a first code for identifying a source animal associated with an animal-based meat product. The program includes instructions for accessing, from digital animal records for animals, a digital animal record corresponding to the source animal associated with the animal-based meat product. The program includes instructions for generating, based on the digital animal record corresponding to the source animal associated with the animal-based meat product, a first code for identifying the source animal, and for. The program includes instructions for updating the digital animal record corresponding to the source animal associated with the animal-based meat product with the first code for identifying the source animal, and for transmitting the first code to a receiving device.

Example 20: The system of Example 19, where the program further includes instructions for: receiving a request to generate a second code for a packaging label associated with the animal-based meat product; accessing, from the digital animal records for animals, a digital animal record corresponding to the source animal associated with the animal-based meat product; generating, based on the digital animal record corresponding to the source animal associated with the animal-based meat product, a second code for accessing health information associated with the source animal of the animal-based meat product; updating the digital animal record corresponding to the source animal associated with the animal-based meat product with the second code; and transmitting the second code to a receiving device.

In certain embodiments, a system includes one or more processors and a non-transitory computer-readable storage medium storing a program to be executed by the processor. The program includes instructions for: generating a user interface comprising fields for managing animal health associated with one or more animals; generate a digital animal record for the one or more animals, the digital animal record comprising health records associated with the one or more animals; and causing the digital animal record to be stored.

In certain embodiments, a non-transitory computer-readable storage medium stores a program to be executed by one or more processors. The program includes instructions for: generating a user interface comprising fields for managing animal health associated with one or more animals; generating a digital animal record for the one or more animals, the digital animal record comprising health records associated with the one or more animals; and causing the digital animal record to be stored.

In certain embodiments, a method includes: generating a user interface comprising fields for managing animal health associated with one or more animals; generating a digital animal record for the one or more animals, the digital animal record comprising health records associated with the one or more animals; and causing the digital animal record to be stored.

In certain embodiments, a system includes one or more processors and a non-transitory computer-readable storage medium storing a program to be executed by the processor. The program includes instructions for: accessing a digital data record for an animal, the digital data record comprising health records associated with the animal; and generating a machine-readable image file that, when decoded, comprises a link to information determined from the digital data record, the information comprising a source of the animal and one or more health facts associated with the animal. In certain embodiments, a physical representation of the machine-readable image file comprises a QR code or a barcode.

In certain embodiments, a non-transitory computer-readable storage medium stores a program to be executed by one or more processors. The program includes instructions for: accessing a digital data record for an animal, the digital data record comprising health records associated with the animal; and generating a machine-readable image file that, when decoded, comprises a link to information determined from the digital data record, the information comprising a source of the animal and one or more health facts associated with the animal. In certain embodiments, a physical representation of the machine-readable image file comprises a QR code or a barcode.

In certain embodiments, a method includes: accessing a digital data record for an animal, the digital data record comprising health records associated with the animal; and generating a machine-readable image file that, when decoded, comprises a link to information determined from the digital data record, the information comprising a source of the animal and one or more health facts associated with the animal. In certain embodiments, a physical representation of the machine-readable image file comprises a QR code or a barcode.

An example processing system, according to certain embodiments of this disclosure, may be configured to perform methods/operations described in this disclosure, and may be installed in a host device. The processing system may include a processor, a memory, and interfaces. The processor may be any component or collection of components adapted to perform computations and/or other processing related tasks, and the memory may be any component or collection of components adapted to store programming and/or instructions for execution by processor. In an embodiment, the memory includes a non-transitory computer readable medium. The computer-readable non-transitory media includes all types of computer readable media, including magnetic storage media, optical storage media, and solid-state storage media and specifically excludes signals. It should be understood that the software can be installed in and sold with the device. Alternatively, the software can be obtained and loaded into the device, including obtaining the software via a disc medium or from any manner of network or distribution system, including, for example, from a server owned by the software creator or from a server not owned but used by the software creator. The software can be stored on a server for distribution over the Internet, for example.

The architecture described herein may be implemented in a computer or network of computers having one or more processors executing instructions of software programs that are stored in one or more computer-readable storage. In some embodiments, the processing system is included in a network device that is accessing, or part otherwise of, a telecommunications network. In one example, the processing system is in a network-side device in a wireless or wireline telecommunications network, such as an applications server or any other device in the telecommunications network. In other embodiments, the processing system is in a user-side device accessing a wireless or wireline telecommunications network, such as a mobile station, a user equipment (UE), a personal computer (PC), a tablet, a wearable communications device (e.g., a smartwatch, etc.), or any other device adapted to access a telecommunications network.

While this disclosure has been described with reference to illustrative embodiments, this description is not intended to be construed in a limiting sense. Various modifications and combinations of the illustrative embodiments, as well as other embodiments of the disclosure, will be apparent to persons skilled in the art upon reference to the description. It is therefore intended that the appended claims encompass any such modifications or embodiments.

What is claimed is:

1. A non-transitory computer-readable storage medium storing a program for execution by one or more processors, the program including instructions for:
receiving a label-generating request for generating a machine-readable image file for an animal-based meat product, the animal-based meat product corresponding to a particular source animal;
accessing, in response to receiving the label-generating request, digital animal records for a plurality of source animals to identify a digital animal record for the particular source animal corresponding to the animal-based meat product, the digital animal records comprising animal information for the plurality of source animals, the animal information comprising identification information, location information, and health information for the plurality of source animals;
generating, based on the animal information of the digital animal record for the particular source animal of the animal-based meat product, a machine-readable image file for the particular source animal of the animal-based meat product, wherein the machine-readable image file, when decoded, makes available meat facts regarding the particular source animal of the animal-based meat product; and
transmitting the machine-readable image file to a requesting device.

2. The non-transitory computer-readable storage medium of claim 1, wherein the machine-readable image file, when decoded, comprises a website address to a website that provides the meat facts about the particular source animal of the animal-based meat product based at least in part on an animal identifier.

3. The non-transitory computer-readable storage medium of claim 2, wherein the program further includes instructions for:
receiving an information request from a user device that has decoded the website address from an optical scan of a package label that includes a physical representation of the machine-readable image file, the information request providing the animal identifier, the animal identifier corresponding to the particular source animal;
accessing, according to the animal identifier and from the digital animal records for the plurality of source animals, the health information for the particular source animal;
generating, according to the health information for the particular source animal, data for receipt by the user device, the data comprising the meat facts regarding the particular source animal; and
transmitting the data to the user device for display.

4. The non-transitory computer-readable storage medium of claim 1, wherein the machine-readable image file, when decoded, comprises the meat facts about the particular source animal of the animal-based meat product.

5. The non-transitory computer-readable storage medium of claim 1, wherein the meat facts comprise one or more of:
information regarding vaccinations of the particular source animal;
information regarding antibiotics of the particular source animal;
information regarding respiratory health of the particular source animal;
information regarding liver health of the particular source animal; or
information regarding neurological health of the particular source animal.

6. The non-transitory computer-readable storage medium of claim 1, wherein the program further includes instructions for:
receiving, after a sale of the animal-based meat product, purchaser data that comprises purchaser-identifying information and a date of purchase; and
updating the digital animal records with the purchaser data so that a sale of the animal-based meat product is linked to a particular purchaser.

7. The non-transitory computer-readable storage medium of claim 6, wherein the program further includes instructions for:
receiving a request to determine location information associated with the particular source animal for animal-based meat product, the request comprising a purchaser identifier for a purchaser of the animal-based meat product;
accessing, according to the purchaser identifier and the purchaser-identifying information of the purchaser data, the digital animal record for the particular source animal of the animal-based meat product to determine location information for the particular source animal; and
transmitting the location information for the particular source animal.

8. The non-transitory computer-readable storage medium of claim 1, wherein the program further includes instructions for:
generating, according to the animal information, an animal health score for the particular source animal; and
updating the digital animal records with the animal health score, the meat facts comprising the animal health score.

9. The non-transitory computer-readable storage medium of claim 1, wherein:
the animal-based meat product is an animal carcass from which multiple sub-products are to be formed; or
the animal-based meat product is a meat sub-product formed from an animal carcass.

10. The non-transitory computer-readable storage medium of claim 1, wherein the particular source animal is a cow or a pig.

11. The non-transitory computer-readable storage medium of claim 1, wherein:
the machine-readable image file that is transmitted to the requesting device is a physical representation of the machine-readable image file that is printable; and
the physical representation of the machine-readable image file comprises one or more of a quick resource (QR) code or a barcode.

12. A method, comprising:
decoding, by a user device, data coded in an optical scan of a physical representation of a machine-readable image file located on a label for an animal-based meat product; and
accessing, by the user device according to the data coded in the optical scan, meat facts about a particular source animal corresponding to the animal-based meat product; and
displaying, by the user device, the meat facts about the particular source animal associated with the animal-based meat product.

13. The method of claim 12, wherein:
the physical representation of the machine-readable image file comprises one or more of a quick resource code (QR code) or a barcode, the data coded in the optical scan being coded in the QR code or barcode and comprising the meat facts;
the data coded in the optical scan of the label comprises the meat facts about the source animal associated with the meat product; and
accessing, by the user device according to the data coded in the optical scan, the meat facts about the particular source animal corresponding to the animal-based meat product comprises determining the meat facts directly from the data coded in the optical scan of the physical representation of the machine-readable image file.

14. The method of claim 13, wherein the meat facts are determined by a processing system distinct from the user device and having access to digital animal records for a plurality of source animals, the plurality of source animals comprising the particular source animal such that the digital animal records comprises a particular data record corresponding to the particular source animal, the processing system having generated the machine-readable image file according to the particular data record corresponding to the particular source animal.

15. The method of claim 12, wherein:
the data coded in the optical scan of the label comprises:
an identifier for mapping to a digital animal record for the particular source animal for the animal-based meat product; and
a website address; and
accessing, by the user device according to the data coded in the optical scan, the meat facts about the particular source animal corresponding to the animal-based meat product comprises:
initiating transmission of a request using the website address, the request comprising the identifier; and
receiving, in response to the request, the meat facts about the particular source animal corresponding to the animal-based meat product.

16. The method of claim 15, wherein the meat facts are received from a processing system distinct from the user device, the processing system having determined the meat facts by accessing digital animal records for a plurality of source animals and determining the meat facts for the particular source animal according to the identifier.

17. The method of claim 15, wherein the website address comprises a Uniform Resource Location (URL).

18. The method of claim 12, wherein the meat facts comprise health information associated with the particular source animal.

19. A system, comprising:
a storage system that stores a plurality of digital animal records for a plurality of animals, the plurality of digital animal records comprising identifying information for the plurality of animals and animal health information for the plurality of animals;
one or more processors; and
a non-transitory computer-readable storage medium storing a program for execution by the one or more processors, the program including instructions for:
receiving a request to generate a first code for identifying a source animal associated with an animal-based meat product;
accessing, from digital animal records for a plurality of animals, a digital animal record corresponding to the source animal associated with the animal-based meat product;
generating, based on the digital animal record corresponding to the source animal associated with the animal-based meat product, a first code for identifying the source animal;
updating the digital animal record corresponding to the source animal associated with the animal-based meat product with the first code for identifying the source animal; and
transmitting the first code to a receiving device.

20. The system of claim 19, wherein the program further includes instructions for:
receiving a request to generate a second code for a packaging label associated with the animal-based meat product;
accessing, from the digital animal records for a plurality of animals, a digital animal record corresponding to the source animal associated with the animal-based meat product;
generating, based on the digital animal record corresponding to the source animal associated with the animal-based meat product, a second code for accessing health information associated with the source animal of the animal-based meat product;
updating the digital animal record corresponding to the source animal associated with the animal-based meat product with the second code; and
transmitting the second code to a receiving device.
